(19) United States
(12) Patent Application Publication
(10) Pub. No.: US 2005/0281904 A1
(43) Pub. Date: Dec. 22, 2005

(54) Kefir extract as an anti-cancer agent

(75) Inventors: Stan Kubow, Pointe-Claire (CA); Laurie Hing Man Chan, Baie D'Urfe (CA); Chujian Chen, Jiangsu Nantong (CN); Maryam Fotouhinia, Montreal (CA)

Correspondence Address: WOOD, PHILLIPS, KATZ, CLARK & MORTIMER, 500 W. MADISON STREET, SUITE 3800, CHICAGO, IL, 60661, US

(73) Assignee: McGill University

(21) Appl. No.: 11/099,731

(22) Filed: Apr. 6, 2005

Related U.S. Application Data
(63) Continuation-in-part of application No. 10/311,504, filed on Jul. 14, 2003, now abandoned.
(60) Provisional application No. 60/211,804, filed on Jun. 16, 2000.

(51) Int. Cl. A61K 35/68

(52) U.S. Cl. 424/780

(57) ABSTRACT

An anti-cancer or prophylactic composition having neutraceutical properties which comprises a filtrated bacteria free and/or yeast free liquid extract of initial fermentative kefir in association with a pharmaceutically acceptable carrier and methods of use are disclosed.

### CROSS REFERENCE TO RELATED APPLICATIONS

This application in a continuation-in-part of U.S. application Ser. No. 10/311,504, filed Dec. 16, 2002, which is a §371 application of PCT/CA01/00896, filed Jun. 15, 2001 which claims the priority of U.S. 60/211,804 filed Jun. 16, 2000.

### BACKGROUND OF THE INVENTION

(a) Field of the Invention

The invention relates to novel anti-cancer agent and uses thereof in cancer treatment.

b) Description of Prior Art

Research on the putative health benefits of fermented milks (FM) has grown dramatically in the past 20 years. In particular, by-products of bacterial fermentation of proteins, lipids and carbohydrates present in FM have been implicated to exert health benefits beyond basic nutrition including anti-tumor action, immune system enhancement and antioxidant effects. Epidemiological studies have indicated a reduced risk of breast cancer in women who consumed FM products (Veer P. et al. Cancer Res 49:4020-4023, 1989). Antimutagensis of FM has also been widely demonstrated (Abdelali H. et al., Mutation Res 331:133-141, 1995). The active ingredients in the fermented milk products have not been fully characterized but several studies suggested that the antimutagenic effect of these cultured milk was due to the presence of the lactic acid bacteria (Pol-Zobel B L et al., Nutr Cancer 20:261-270, 1993). Abdelali (Abdelali H. et al., Mutation Res 331:133-141, 1995) reported that the bifidobacterium sp., casein and calcium components in FM showed a dose-dependent antimutagenic activity against benzo[a]pyrene mutagenicity in the Ames test using Salmonella typhimurium TA98. In animal models, lactobacilli and bifidobacteria have been shown to inhibit the growth or cause regression of tumors, which have been transplanted or chemically induced. Shiomi et al. (Shiomi M. et al., Jap J Med Sci Bio. 35:75-80, 1982) showed that polysaccharides extracted from kefir grain had antitumor activity in mice.

The FM product, kefir, enjoys a rich tradition of health claims, as consumption of kefir has been used in the former Soviet Union for the treatment of a variety of conditions including metabolic disorders, atherosclerosis, cancer, and gastrointestinal disorders (Koroleva N S. IDF Bull. 227:35-40, 1988). In the former Soviet Union, kefir accounts for 70% of the total amount of FM consumed. Kefir distinguishes itself from the more known FM product, yogurt, in that it is traditionally made only from kefir grains which contain a complex mixture of both bacteria and yeast. Hence, in kefir production the milk undergoes a dual fermentation process under the action of both lactic acid bacteria and yeasts.

While yogurt can readily be made from the lactic acid bacteria present in fresh yogurt, kefir can only be made from kefir grains and mother cultures prepared from grains. The grains contain a relatively stable and specific balance of microorganisms, which exist in a complex symbiotic relationship. The grains are formed in the process of making kefir and only from pre-existing grains. The grains include primarily lactic acid bacteria (lactobacilli, lactococci, leuconostocs) and yeast. They resemble small cauliflower florets, and each grain is 3 to 20 mm in diameter. Kefir grains are clusters of microorganisms held together by a matrix of polysaccharides. Kefiranofaciens and L. kefir produce these polysaccharides. The polysaccharides are an integral part of the grain, and without their presence, kefir grains cannot be propagated.

Encouraging results regarding an anti-tumor activity of kefir in animal studies have been reported (Shiomi M. et al., Jap J Med Sci Bio. 35:75-80, 1982; Cevikbas A et al., Phytother Res 8:78-82, 1994; Furukawa N et al., J Jap. Soc. Food Sci. 43:450-453, 1990; Kubo M. et al., Pharmacological study on kefir—a fermented milk product in Cancasus. I. On antitumor activity (1) Yakugaku Zasshi 112:489-495, 1992). For example, oral doses of 100 or 500 mg/kg of kefir to mice with solid tumor of E-ascites carcinoma (EC) transplanted s.c. were shown to cause a significant reduction in transplanted tumor size and activate the immunosuppressive activity of the spleen (Kubo M. et al. Phamacological study on kefir—a fermented milk product in Cancasus. I. On antitumor activity (1) Yakugaku Zasshi 112:489-495, 1992).

It has been reported that modulation of cellular glutathione (GSH) concentrations has been indicated to play a crucial role in cell proliferation and tumor resistance. Indeed, numerous studies have demonstrated that cellular concentrations of GSH respond differentially to GSH-modulating treatments in normal versus tumor cells lines, which also show major differential outcomes from these treatments in terms of cellular viability and proliferation (Russo et al., 1986; Roberts et al., 1991; Baruchel and Viau, 1996).

It has also been shown that cellular GSH-depletion results in a reduced rate of cell proliferation in human lung (Kang Y J, Enger M D. Glutathione content and growth in A549 human lung carcinoma cells. Exp Cell Res 1990; 187: 177-79) and colon carcinoma cells (Benard O, Balasubramanian K A. Modulation of glutathione level during butyrate-induced differentiation in human colon derived HT-29 cells. Mol Cell Biochem 1997; 170: 109-14).

Moreover, findings in terms of selective effects on the proliferation and cellular GSH levels of tumor cells have been reported with whey protein concentrates (WPC) and isolates. Treatment with WPC has been shown to inhibit rat mammary tumor or Jurkat T tumor cell proliferation (Baruchel and Viau, 1996). The authors speculated that the selective inhibition of GSH in tumor vs. normal cells was a possible mechanism explaining the antiproliferative effect of WPC since GSH levels were decreased with WPC treatment only in tumor cells. Supportive evidence for the functional significance of the selective modulation of tissue GSH and the therapeutic anti-cancer properties of WPC has also come from animal experiments, which have shown that WPC exhibit anticancer activities via modulation of the GSH pathway, the induction of p53 protein in transformed cells and inhibition of neoangiogenesis (Bounous G. Molson J H. The antioxidant system. Anticancer Research. 23(2B):1411-5, 2003). Animal studies have shown that whey protein, in comparison to red meat, soy bean meal and/or casein, is more protective against carcinogen-induced colon tumor expression (Bounous, R. Papenburg, P. A. Kongshavn, P. Gold and D. Fleiszer, Dietary whey protein inhibits the development of dimethylhydrazine induced malignancy. Clin. Invest. Med. 11 (1988), pp. 213-217; R. Papenburg, G. Bounous, D. Fleiszer and P. Gold, Dietary milk proteins inhibit the development of dimethylhydrazine-induced malignancy. Tumour Biol. 11 (1990), pp. 129-136; G. H. McIntosh, G. O. Regester, R. K. Le Leu, P. J. Royle and G. W. Smithers, Dairy proteins protect against dimethylhydrazine-induced intestinal cancers in rats. J. Nutr. 125 (1995), pp. 809-816 R. Hakkak, S. Korourian, M. J. Ronis, J. M. Johnston and T. M. Badger, Dietary whey protein protects against azoxymethane-induced colon tumors in male rats. Cancer Epidemiol. Biomark. Prev. 10 (2001), pp. 555-558). In addition, dietary 3 0 whey protein hydrolysates administered throughout development prevent 7,12-dimethylbenz(a)anthracene (DMBA)-induced mammary cancers in adult female Sprague-Dawley rats (Hakkak, R., Korourian, S., Shelnutt, S. R., Lensing, S., Ronis, M. J., and Badger, T. M. (2000). Diets containing whey proteins or soy protein isolate protect against 7,12-dimethylbenz(a)anthracene-induced mammary tumors in female rats. Cancer Epidemiol. Biomarkers Prev. 9, 113-117). The chemotherapeutic importance of depletion of tumor cell GSH involving WPC treatment has been indicated by a phase II clinical trial involving daily intake of WPC in patients with metastatic carcinoma of the breast, pancreas and liver. The patients initially showed above normal blood lymphocyte GSH levels reflecting high tumour GSH levels. Patients that responded with lowered blood lymphocyte GSH levels following WPC treatment, however, showed stabilization of the tumour growth (Kennedy R S, Konok G P, Bounous G, Baruchel S, Lee T D. The use of a whey protein concentrate in the treatment of patients with metastatic carcinoma: a phase I-II clinical study. Anticancer Res. 1995 15:2643-9). Another study showed that a reduction of cellular GSH concentrations by 20% in human hepatoma HepG2 cells treated with a whey protein isolate enhanced significantly the cytotoxic action of the anticancer drug, baicalein on HepG2 cells (Tsai et al. Nutr. Cancer 2000 38: 200-208).

Recent evidence has suggested a role for aberrantly low levels of cellular ceramide in the pathogenesis and chemoresistance of cancer thereby indicating that enhancement of tumor ceramide levels may be a useful strategy for breast cancer treatment. Ceramide is derived from sphingomyelin, and can act as an intracellular second messenger for tumor necrosis factor-alpha, IL-1beta, and other cytokines. Ceramide has also been implicated in the acquired drug resistance that often characterizes breast cancer cells (Liu et al., 1999). In this regard, ceramide analogs have shown considerable potential in the induction of antiproliferative effects in breast cancer cells depending on the ceramide compound used (Struckhoff et al., 2004). Bansode et al. (2004) reported that incubation of milk-based C6 ceramide in MCF-7 cells at 1 μM decreased cell migration, inhibited cell proliferation, caused cell death by apoptosis, and reduced the levels of angiogenesis stimulators VEGF and cathepsin D to below 50% of the control.

In particular, it is not evident from previous work: (1) whether kefir exerts an anti-proliferative effect on tumor cells and if this effect is specific to tumor cells; and (2) whether there are different anti-proliferative potencies associated with specific stages of kefir manufacture.

It would be highly desirable to be provided with a novel anti-cancer agent and uses thereof in cancer treatment.

### SUMMARY OF THE INVENTION

One aim of the present invention is to provide a novel anti-cancer agent and uses thereof in cancer treatment.

In accordance with the present invention there is provided an anti-cancer composition having anti-proliferative and/or inhibitory effects specifically targeted at malignant cells, which comprises a filtrated bacteria-free and/or yeast-free liquid extract of initial fermentative kefir in association with a pharmaceutically acceptable carrier.

The filtrated extract of the anti-cancer composition in accordance with a preferred embodiment of the present invention, is ultrafiltrated or microfiltrated.

The liquid extract of the anti-cancer composition in accordance with a preferred embodiment of the present invention, comprises a protein concentration of 300 ng/ml to 5000 ng/ml, or more preferably of about 313 ng/ml.

In accordance with the present invention there is also provided a method of inhibiting proliferation of malignant cells in patient, which comprises administering an effective amount of a filtrated bacteria-free and/or yeast-free liquid extract of initial fermentative kefir.

The malignant cells used in a method in accordance with a preferred embodiment of the present invention, are selected from the group consisting of estrogen responsive cancer, such as breast or uterine cancer, cancer induced by oncovirus, hepatic cancer, colon cancer, prostate cancer, skin cancer and lung cancer.

In accordance with the present invention there is also provided a prophylactic composition having neutraceutical properties, which comprises a filtrated bacteria-free and/or yeast-free liquid extract of initial fermentative kefir in association with a pharmaceutically acceptable carrier.

For the purpose of the present invention the following terms are defined below.

The term “kefir” is intended to mean an end-product of a kefir manufacturing process.

The term “liquid extract of initial fermentative kefir” is intended to mean an intermediate fermentation by-product of a kefir manufacturing process.

### BRIEF DESCRIPTION OF THE DRAWINGS

FIGS. 1A and 1B illustrate the effects of different extracts from different stages in the manufacture of kefir (FIG. 1A) and yogurt (FIG. 1B) on MCF-7 cells;

FIGS. 2A and 2B illustrate the effects of different extracts from different stages in the manufacture of kefir (FIG. 2A) and yogurt (FIG. 2B) on HMEC normal human mammary epithelial cells; and

FIG. 3 illustrates a schematic representation of the kefir manufacture.

FIG. 4 illustrates the effect of different doses of Kefir extracts on intracellular glutathione (GSH) concentrations (Mean±SEM) in MCF-7 cells.

FIG. 5 illustrates the effect of different doses of Kefir extracts on intracellular glutathione (GSH) concentrations (Mean±SEM) in HMEC cells.

FIG. 6 illustrates the antiproliferative effect of RP-HPLC Fraction 30 of kefir with or without tamoxifen on MCF-7 cells.

FIGS. 7A and 7B illustrate the antiproliferative effects of extract of kefir (A) and MWCO less than 3000 Da (B) with or without ceramide I on MCF-7 cells.

FIGS. 8A and 8B illustrate the antiproliferative effects of extract of kefir (A) and fraction of MWCO less than 3000 Da (B) with or without ceramide 1-phosphate on MCF-7 cells.

FIGS. 9A and 9B illustrate the antiproliferative effects of extract of kefir (A) and fraction of MWCO less than 3000 Da (B) with or without C6 ceramide on MCF-7 cells.

FIGS. 10A to 10D illustrate the dose effect of preparative RP-HPLC Fraction 29 (A), 30 (B), 34 (C), and 37 (D) on MCF-7 and HMEC cells.

FIGS. 11A and 11B illustrate the comparison of TNF-alpha concentration in cell culture medium after MCF-7 cells (A) and HMEC cells (B) were treated with whole extract of kefir, SEC HPLC fraction 7 of kefir extract, and preparative HPLC fraction 30 of kefir extract.

### DETAILED DESCRIPTION OF THE INVENTION

Surprisingly, and in accordance with the present invention, there is demonstrated for the first time that a fraction of an early stage of kefir manufacture is associated with the most potent anti-proliferative effect on tumor cells. The kefir liquid fraction is a filtrated fraction of the mother culture as illustrated on FIG. 3. This filtrated fraction is substantially free of any bacteria and/or yeast.

Epidemiological studies have indicated that consumption of fermented milk products reduced risk of breast cancer. Effects of kefir, a traditional fermented milk product, on the growth of human mammary cancer cells have not been characterized. Both kefir and yogurt were filtered to eliminate microbes and the extracts were then incubated with normal human mammary epithelial cells and human mammary cancer (MCF-7) cells to examine their effects on cell proliferation. Both kefir and yogurt suppressed the proliferation of human MCF-7 cancer cells but the antiproliferative effects of kefir were significantly greater (p<0.01). After 8 days of culture, the kefir extract (1:640 dilution in medium) decreased MCF-7 cell numbers by 40% while yogurt extract (1:160 dilution in medium) decreased the cell numbers by only 15%. The antiproliferative effects of the two fermented milks were not accountable by lactic acid concentrations in the fermented milk extracts and were not observed in the normal human mammary epithelial cells. Milk extract had no effect on the growth of either the MCF-7 cells or the normal human mammary epithelial cells. These results indicate that kefir and yogurt extracts contain active ingredients that have antiproliferative properties on human mammary cancer cells. Unlike yogurt extracts, the kefir extracts did not suppress the growth of normal human mammary cells suggesting that the kefir extracts contain bioactive ingredients that exert a growth suppressive effect that is specific to cancer cells.

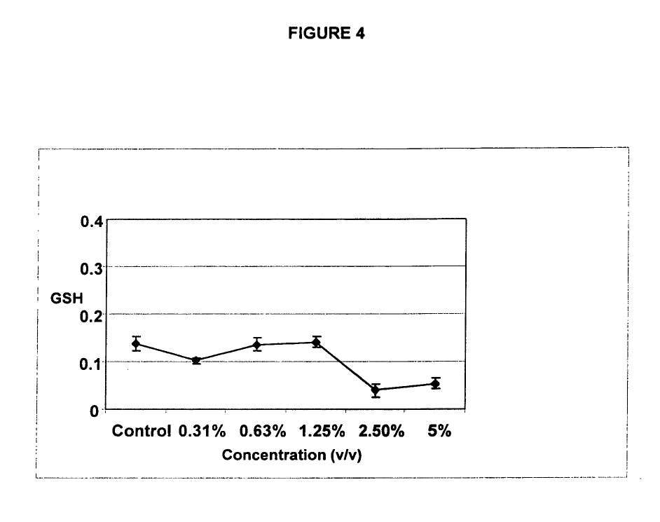

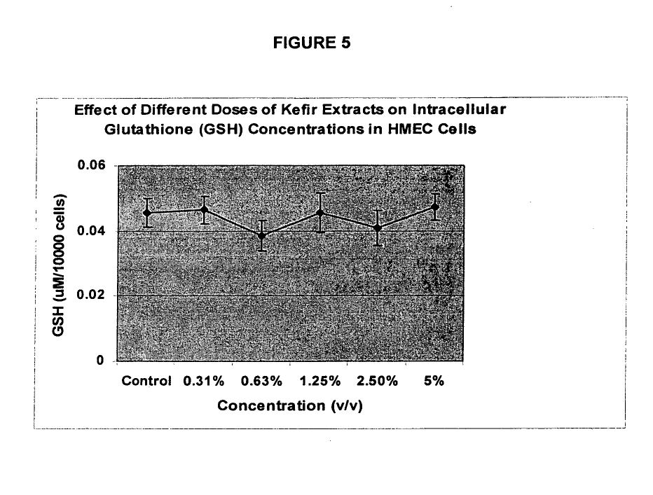

Further basic research has been conducted by the inventors in order to show the possible mechanisms for the reported antiproliferative effects of the kefir extract. As one skilled in the art may appreciate, the whole extract of kefir was associated with significantly lower intracellular GSH concentrations (P<0.05) in MCF-7 tumor cells (FIG. 4). Notably, whole kefir extract was not associated with changes in cellular GSH concentrations in normal human mammary epithelial cells (HMEC) cells (FIG. 5). The selective depletion of intracellular GSH in MCF-7 cells associated with whole kefir extract treatment is a plausible mechanism by which whole kefir extract exerts anti-proliferative effects on cancer cells either singly or in combination with anti-cancer agents such as tamoxifen or ceramides.

The depletion of GSH by whole kefir extract that is specific to MCF-7 tumor cells could thus be a therapeutic approach as well as a useful adjunct to chemotherapy since elevated GSH levels in tumor tissue are associated with resistance to chemotherapy (Schröder C P, Godwin A K, O'Dwyer P J, Tew K D, Hamilton T C, Ozols R F. Glutathione and drug resistance. Cancer Invest 1996; 14: 158-68).

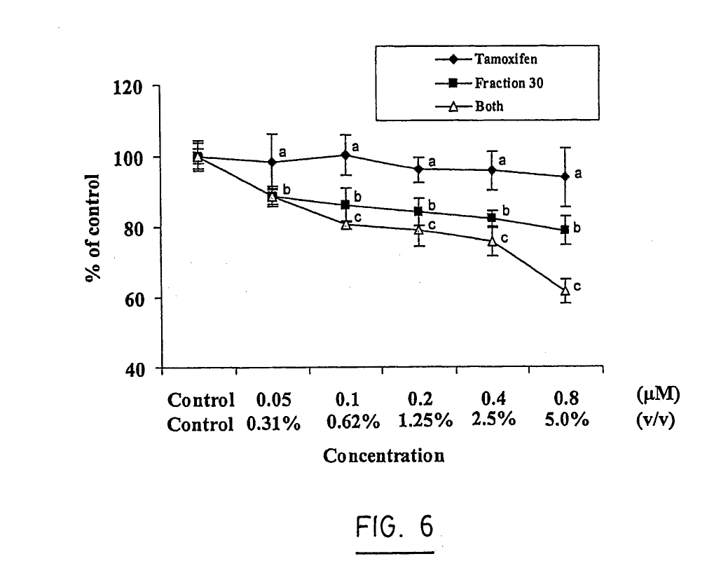

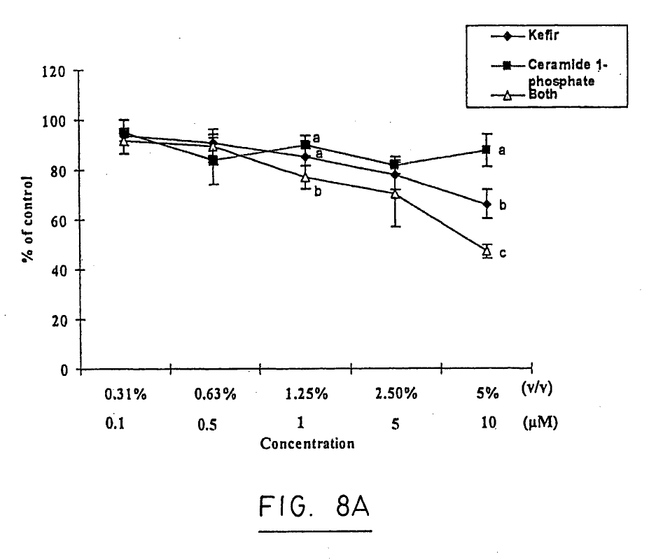

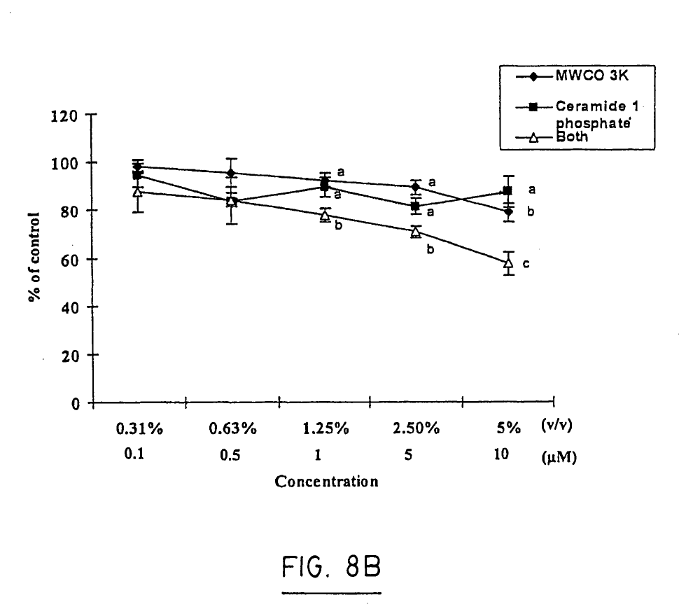

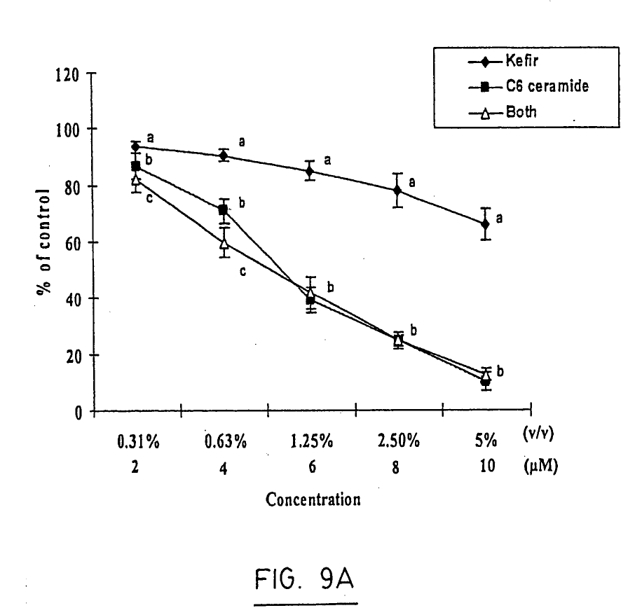

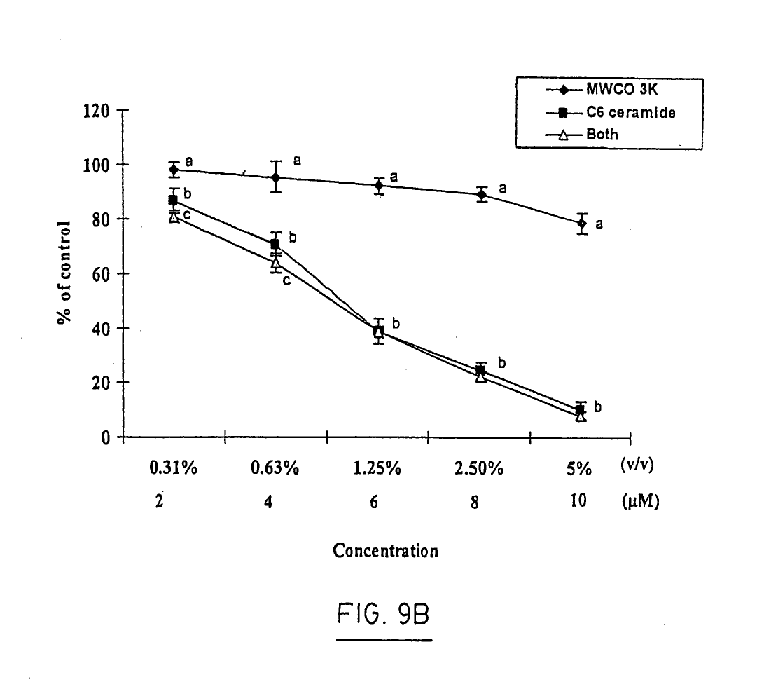

In that regard, the inventors show therein that the combination of kefir extract with anti-cancer agents such as tamoxifen or ceramide compounds enhanced their cytotoxicity in MCF-7 cells but not in HMEC cells (FIGS. 6-9).

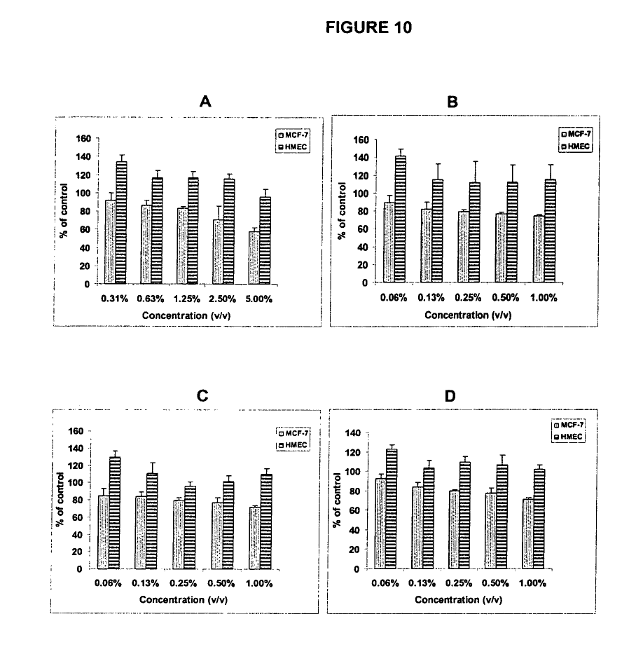

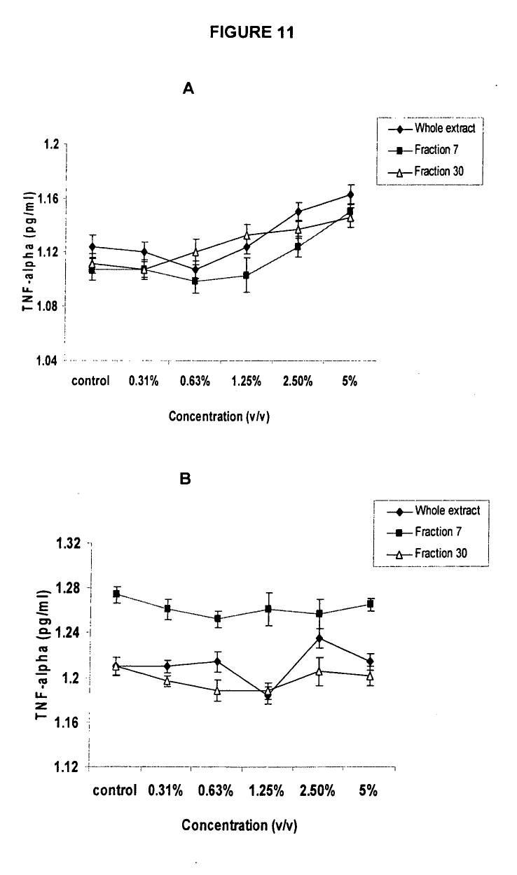

Tamoxifen is a commonly used medication for breast cancer patients. In the present study, dosages above 0.2 μM/L showed antiproliferative effect on MCF-7 cells. This effective dose is higher than the value reported by Doisneau-Sixou et al. (2003). A preparative C4 column was utilized to obtain sufficient amounts of mother culture kefir fractions of MWCO less than 3000 Da for more extensive structure analysis and cell culture studies. The average nitrogen content of the lyophilized filtrate of MWCO less than 3000 Da was 0.58%. Five g of the lyophilized molecular weights less than 3000 Da fractions was dissolved in 20 mL of water. Ten milliliters of reconstituted solution were loaded on the column, and 100 fractions were collected in 1 min interval. A dose dependent antiproliferative effect was observed when MCF-7 cells were treated with fractions 29, 30, 34, and 37, while not on HMEC cells (FIGS. 10A to D). The reverse-phase-HPLC Fraction 30 of kefir mother culture extract increased significantly the susceptibility of MCF-7 cells to tamoxifen (FIG. 6). Elevated TNF-alpha levels have been observed after the MCF-7 cells were treated with whole kefir extract or kefir fractions (FIG. 11A). TNF-alpha in conjunction with tamoxifen produced synergistic inhibition of MCF-7 cells in vitro (Brosellino et al., 1994; Saftri and Bonavida 1992; Cimoli et al., 1993) indicating the potential of using the kefir extract as co-drug for chemotherapy.

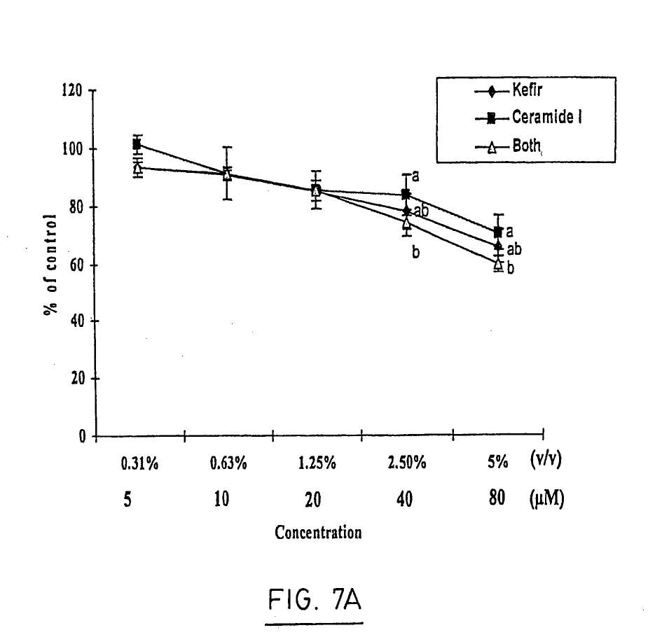

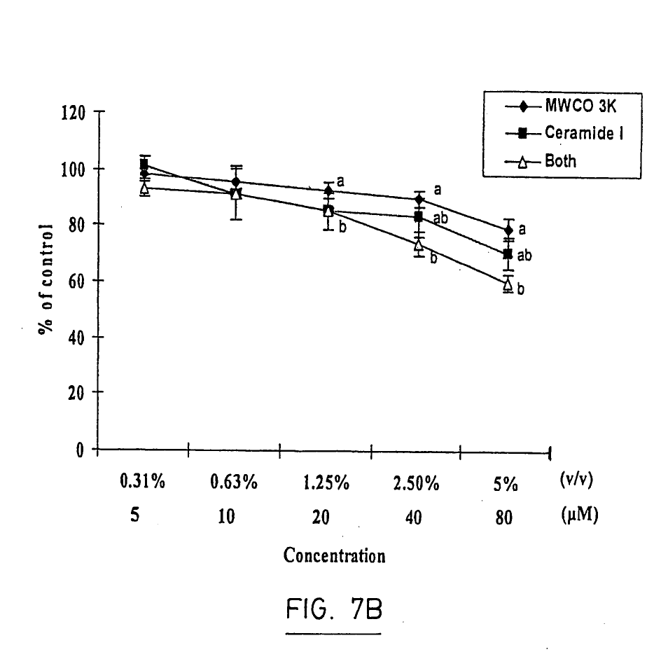

The present results demonstrate that ceramide I (C2-ceramide analog) and C6 ceramide showed a strong dose dependent antiproliferative effect on MCF-7 cells (FIGS. 7-9) while ceramide 1-phosphate (C18 ceramide analog) from bovine brain did not. This result is similar to the findings of Fillet et al. (2003) who observed that C2 and C6 ceramides, as cell permeable ceramide analogs, induced apoptosis in HCT116 and OVCAR-3 cancer cells whereas C16-ceramide, which is impermeable, showed no effect. Ceramide analogs are indicated to induce apoptosis via nuclear factor-κBDNA-binding, caspase-3 activation, poly(ADP-ribose) polymerase degradation, and mitochondrial cytochrome c release, indicating that apoptosis occurs through the caspase cascade and the mitochondrial pathways. The inventors observed a synergistic effect when MCF-7 cells were treated with ceramide analogs in the presence of extract of whole kefir (FIGS. 7-9).

Although whey proteins were isolated in the milk extract, no anti-cancer activities were noted as opposed to the kefir-fermented milk extract. It is likely that the dose of whey proteins provided by the milk extract was too low to induce anti-proliferative action. On the other hand, fermentation induced antiproliferative effects as kefir fermented milk extracts showed a potent antiproliferative effects on tumor cells. Treatment of MCF-7 cells via fermented milk in the form of yogurt extracts showed significantly lower anti-proliferative potency relative to the kefir extract. Also, as opposed to kefir extract treatment, anti-proliferative effects were observed in normal mammary cells following yogurt extract treatment. Hence, as has been generally observed with WPC treatment in normal and tumor cell lines, the whole kefir extract potent anti-proliferative effects on MCF-7 cells but not in normal mammary epithelial cells. This latter result is unexpected in light of findings that other fermented cow's milk products such as yogurt have typically shown to exert antiproliferative effects on both tumor cell lines such as gut-derived adenocarcinoma Caco-2 cells as well as normal non-transformed cell lines such as the rat small intestinal epithelial cell line IEC-6 (Thoreux K, Senegas-Balas F, Bernard-Perrone F, Giannarelli S, Denariaz G, Bouley C, Balas D. Modulation of proliferation, second messenger levels, and morphotype expression of the rat intestinal epithelial cell line IEC-6 by fermented milk. J Dairy Sci. January 1996;79(1):33-43; Ganjam, L. S., Thornton, W. H. Jr, Marshall, R. T. and Macdonald, R. S. (1997). Antiproliferative effects of yogurt fractions obtained by membrane dialysis on cultured mammalian intestinal cells. J. Dairy Sci. 80(10): 2325-9).

Thus, the results on kefir extracts show:

(a) antiproliferative and GSH depleting effects that are specific to tumor cells;

(b) the enhancement of the cytotoxicity of anticancer agents support strongly the idea that similar physiological mechanisms of action are involved with kefir extract and WPC treatments, which are not observed with milk or yogurt extracts.

Further evidence of the therapeutic importance of whole kefir extracts was the induction of TNF-alpha in the MCF-7 tumor cells, which was not observed in the HMEC cells (FIG. 11). In addition to GSH, one of the prominent factors involved in tumor cell proliferation is TNF-alpha, which causes the induction of apoptosis in a variety of tumor cells (C. F. Ware, S. VanArsdale and T. L. VanArsdale, Apoptosis mediated by the TNF-related cytokine and receptor families. J Cell Biochem 60 (1996), p. 47). Since cellular depletion of glutathione is associated with a greater sensitivity to TNF-alpha-induced apoptosis; Kaipia et al., 1996). The antiproliferative effect of kefir extracts on MCF-7 cells may thus be explained by TNF-alpha induced apoptosis. Interestingly and as shown in the following table, the enhanced TNF-alpha concentrations in MCF-7 cells following whole kefir extract treatment were associated with lowered cellular GSH concentrations.

TABLE 1
Correlation analyses of GSH and TNF-alpha after MCF-7 and
HMEC cells were treated with whole kefir extract

| | | GSH vs | | |
|---|---|---|---|---|
| | | cell | TNF-α vs | GSH vs |
| | | number | cell number | TNF-α |
| Whole kefir | MCF-7 | 0.76* | −0.85* | −0.86* |
| extract | HMEC | 0.13 | 0.22 | 0.34 |

*Significance test of correlation coefficient, P < 0.05

The inverse relationship observed between TNF-alpha and GSH concentrations is plausible since as cytokines such as TNF-alpha act as mediators of oxidative stress and they have been indicated to decrease GSH concentrations by affecting GSH shuttling and recycling (Chen C Y, Huang Y L and Lin T H (1998) Association between oxidative stress and cytokine production in nickel-treated rats. Arch Biochem Biophys 356: 127-132). TNF-alpha can impair GSH-redox status by a variety of mechanisms including increased expenditure of reduced GSH resulting from an enhanced production of reactive oxygen species and diminished GSH reductase activity, which results in decreased regeneration of reduced GSH from the oxidized form (Buttke T M, Sandstrom P A. Oxidative stress as a mediator of apoptosis. Immunol Today 1994; 15:7-10; Iosli H, Tronstad K J, Wergedal H, et al. Human TNF-α in transgenic mice induces differential changes in redox status and glutathione-regulating enzymes. FASEB J 2002; 16:1450-2 Ishii Y, Partridge C A, Del Vecchio P J, Malik A B. Tumor necrosis factor-α-mediated decrease in glutathione increases the sensitivity of pulmonary vascular endothelial cells to H₂O₂. J Clin Invest 1992; 89:794-802). Moreover, induction of TNF-alpha has been associated with depletion of cellular GSH in a variety of tumor cell types such as L929 cells (Hayter et al., 2000). Thus, it appears that induction of TNF-alpha and diminished cellular GSH concentrations in MCF-7 cells are likely mechanisms of action of the anti-proliferative effects of whole kefir extracts of the present invention.

Materials and Methods:

Cell Culture:

MCF7-E3 human breast cancer estrogen-sensitive cells were provided by Dr. D. Desaulniers of Health Canada, Ottawa. Cells were routinely propagated as a monolayer culture in Dulbcco's Modified Eale Medium (DMEM) supplemented with 10% heat-inactivated fetal bovine serum (FBS), in 75-cm² plastic dish at 37° C. in a humidified atmosphere with 5% CO₂, and passage 3-4 days a time. A normal human mammary epithelial cell line was provided by Dr. M. Stampfer of UC Livermore Labs. Cells were routinely propagated as a monolayer culture in Mammary Epithelial Growth Media (MEGM, Clonetics, San Diego) supplemented with 10% heat-inactivated fetal bovine serum (FBS), in 75-cm² plastic dish at 37° C. in a humidified atmosphere with 5% CO₂, and passage every week. For the experiments, both cells were harvest from the dish using 0.25% trypsin-EDTA solution.

Preparation of Extracts:

Four kefir products (K1-K4) collected at various stages of kefir production at Liberty Brand Products, Inc. (Montreal, Canada) were used in this study. The large-scale production of kefir involves a two-step fermentation process. The first step is to prepare the cultures by incubating milk with kefir grains (2-10%) (K1) and fermented for 24 hrs. The grains are then removed by filtration and the resulting mother culture (K2) is added to pasteurized milk (K3), which is further fermented for 12 hrs and this final product (K4) is packaged for the consumer market. A pasteurized milk sample (M1) and two yogurt products; mixture of yogurt bacteria, pasteurized milk and milk powder (Y1) and the final yogurt product after 12 hrs of fermentation (Y2) were included for comparison. The yeast and bacteria in the samples were removed by centrifugation and filtration. About 35 ml each of the seven samples was centrifuged (32,000×g, 60 min, 4° C.) and the supernatant was filtered through a 0.45 μm Millipore filter followed by a 0.2 μm Millipore filter (Millipore Corporation, Bedford, USA). Extracts from two separate batches of kefir and yogurt were used.

Cell Proliferation Experiments:

Cells previously harvested were seeded in 24-well plates; 10,000 cells for MCF-7 per well in DMEM supplemented with 10% FBS and 5,000 cells for HMEC per well in MEGM supplemented with 10% FBS. The cells were allowed to attach for 24 hours. After that period, old media were removed and fresh media and extracts were added to each well. To study the dose response, a serial dilution of each the extract using the culture media was made to achieve final concentrations of extracts at 1:40, 1:80, 1:160, 1:320 and 1:640 (vol/vol) or 2.5%, 1.3%, 0.6%, 0.3% and 0.2% respectively. Because the kefir and yogurt extracts were acidic (pH around 4.5). Dulbecco's Phosphate Buffered Saline (PBS) buffer was added to the culture media to adjust the pH between 7.0-7.6. Cells were incubated at 37° C. in a humidified atmosphere with 5% CO₂ for 8 days and the cell numbers in each well were counted using a Coulter Counter (Coulter Counter Corporation, USA). Each sample was run in quadruplicate. Control cells were incubated with the culture medium with the dosing vehicle (PBS).

Lactic Acid Concentration in Cell Cultural Media, Kefir and Yogurt Extracts:

After cells were collected for counting, the culture media were centrifuged at 4000 rpm for 10 min at 4° C. The supernatant was isolated for lactic acid measurement using a lactic acid assay kit from Sigma Diagnostics Inc. Kefir, milk and yogurt extracts were diluted 10 to 20 times before the measurement of lactic acid.

Statistics:

The cell numbers expressed as percentage of control from different treatments and dose were compared using two-way ANOVA. When the interaction between treatments and dose was also significant the difference between treatment groups was determined by Tukey's HSD multiple comparison test. All statistics test were performed using SAS 6.11 for PC (SAS, Cary, N.C.).

Results

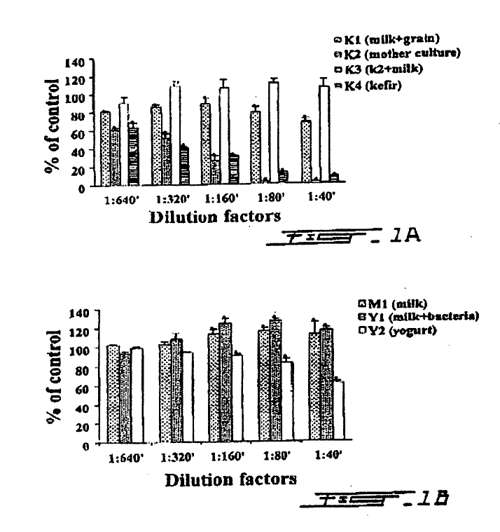

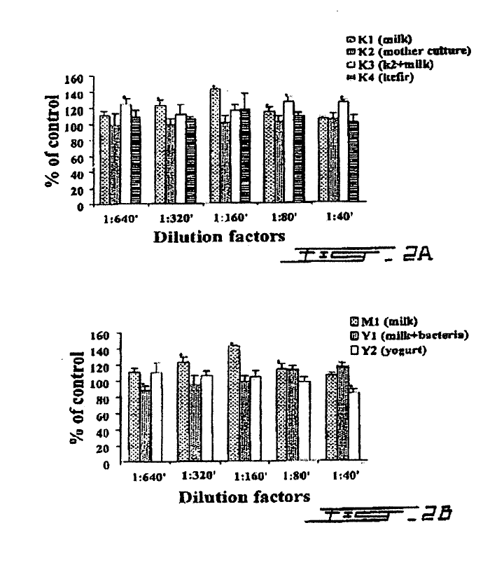

FIG. 1A showed effects of different kefir samples (K1-K4) on proliferation of MCF-7 cells. Values are means±SD (n=4) and *denotes significantly different from control at p<0.01. The effects of both the treatments and dose were significant (p<0.01). The mixture of kefir grain and milk (K1) showed a moderately inhibitory effect (p<0.01), whereas the fermented mother culture (K2) and the final kefir product (K4) showed a significantly stronger inhibitory effect (p<0.01) in comparison to the K1 mixture. Dilution of the mother culture with milk (K3), however, resulted in elimination of inhibitory effects. In addition, the mother culture (K2) had significantly (p<0.05) more potent inhibitory effects on MCF-7 all proliferation than the kefir product (K4) at the 1:80 and 1:40 dilutions.

Yogurt (Y2) also showed similar inhibitory effects on the growth of the MCF-7 cells (FIG. 1B) but the inhibitory effect was significantly less than exhibited by the kefir extracts (p<0.01). Yogurt extract (Y2) at a 1:160 dilution in medium decreased the cell numbers by only 15% whereas kefir extract (K2) at 1:640 dilution in medium decreased the cell numbers by 40%. Milk (M1) and the mixture of milk and yogurt bacteria (Y1) both showed a slight but significant stimulation of cell growth beginning at 1:160 dilution in the media (FIG. 1B).

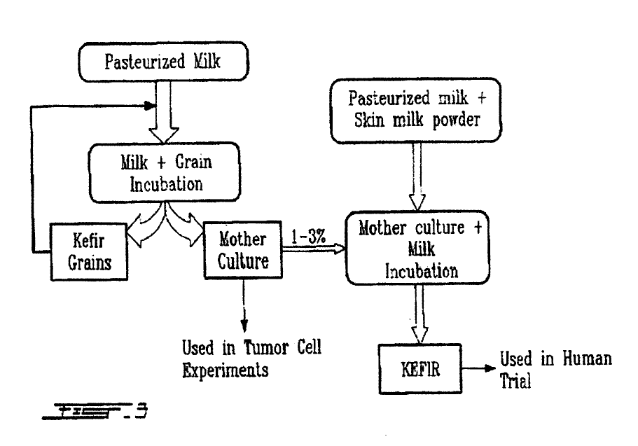

In contrast, both K2 and K4 kefir extracts did not effect the proliferation of the HMEC cells (FIG. 2A). Values are means±SD (n=4) and *denotes significantly different from control at p<0.01. The Y2 yogurt fraction, on the other hand, had a slight inhibitory effect on the growth of the HMEC cells (FIG. 2B) (p<0.05) and lowered the cell number to 83% at a 1:40 dilution in the medium. All the non-fermented milk products (K1, K3, M1 and Y1) showed a slight but significant (p<0.05) stimulation of cell growth (FIGS. 2A & 2B).

Discussion

Previous results have shown that yogurt exert anti-proliferative properties in MCF-7 cells (Biffi A. et al., Nutrition and Cancer 28:93-99, 1997). The present results show, however, that the anti-proliferative potency of kefir extracts on MCF-7 cellular growth is markedly greater than that of yogurt extracts and that, unlike yogurt, the kefir extracts do not suppress the growth of normal human mammary epithelial cells. Thus, this work is the first to indicate that a kefir fraction, unlike other fermented milk products, exerts anti-proliferative effects that are specific to tumor cells. The dose-response concentrations of the extracts used dilutions that varied from 1:640 to 1:40. The antiproliferative activity is clearly not caused by the yeast or bacteria of the kefir or yogurt as the test samples were filter sterilized. Likewise, lactic acid was excluded as the active ingredient since lactic acid measurements showed no relationship with lactic acid concentrations in the test mediums. Thus, the bioactive ingredient(s) must be a fermentation product other than lactic acid. As antiproliferative activity from the kefir extracts is observed in the MCF-7 cells but not the normal human mammary epithelial cells suggest that the active ingredients can bind to or triggers response that are specifically found in tumor cells. Previous work has shown that the administration of a polysaccharide isolated from the kefir grain had anti-tumor activities in mice (Shiomi M et al., Jap J Med Sci Bio. 35:75-80, 1982); however, the polysaccharides show no inhibitory effects against the growth and viability of cultured tumor cells and thus the anti-tumor effects are considered to be host mediated. Thus, the anti-proliferative agent in the kefir extract is unlikely to be a polysaccharide.

Milk proteins and peptides, especially those associated with whey, may be likely candidates as the bioactive ingredients of the kefir extracts. A number of whey proteins have been shown to have anti-carcinogenic properties and incubation of whey protein concentrates have been shown to increase proliferation of normal rat lymphocytes whereas the growth of rat mammary tumor cells was shown to be inhibited (Bourtourault M. et al., CR Soc Biol 185, 319-323, 1991).

The present invention will be more readily understood by referring to the following examples which are given to illustrate the invention rather than to limit its scope.

### EXAMPLE I

### Anti-Cancer Composition

In accordance with one embodiment of the present invention, there is provided a composition having anti-proliferative and/or inhibitory effects specifically targeted at malignant cells which comprises a filtrated bacteria-free and/or yeast-free liquid extract of initial fermentative kefir in association with a pharmaceutically acceptable carrier. This pharmaceutical composition will be administered in a physiologically acceptable medium for oral administration, e.g. deionized water, phosphate buffered saline (PBS), saline, plasma, proteinaceous solutions, aqueous glucose, alcohol, vegetable oil, or the like.

The composition may be lyophilized for convenient storage and transport.

The composition may also be administered parenterally, such as intravascularly (IV), intraarterially (IA), intramuscularly (IM), subcutaneously (SC), or the like. Administration may in appropriate situations be by transfusion. In some instances, administration may be nasal, rectal, transdermal or aerosol.

### EXAMPLE II

### Prophylactic Composition

In accordance with one embodiment of the present invention, there is provided a prophylactic composition having neutraceutical properties, which comprises a filtrated bacteria-free and/or yeast-free liquid extract of initial fermentative kefir in association with a pharmaceutically acceptable carrier.

While the invention has been described in connection with specific embodiments thereof, it will be understood that it is capable of further modifications and this application is intended to cover any variations, uses, or adaptations of the invention following, in,general, the principles of the invention and including such departures from the present disclosure as come within known or customary practice within the art to which the invention pertains and as may be applied to the essential features hereinbefore set forth, and as follows in the scope of the appended claims.

1. An anti-cancer composition having anti-proliferative and/or inhibitory effects specifically targeted at malignant cells, which comprises a filtrated bacteria-free and/or yeast-free liquid extract of initial fermentative kefir in association with a pharmaceutically acceptable carrier.

2. The anti-cancer composition of claim 1, wherein said filtrated extract is ultrafiltrated or microfiltrated.

3. The anti-cancer composition of claim 1, wherein said liquid extract comprises a protein concentration of 300 ng/ml to 5000 ng/ml.

4. The anti-cancer composition of claim 3, wherein said liquid extract comprises a protein concentration of about 313 ng/ml.

5. A method of inhibiting proliferation of malignant cells in patient, which comprises administering an effective amount of a filtrated bacteria-free and/or yeast-free liquid extract of initial fermentative kefir.

6. The method of claim 5, wherein said filtrated extract is ultrafiltrated or microfiltrated.

7. The method of claim 5, wherein said liquid extract comprises a protein concentration of 300 ng/ml to 5000 ng/ml.

8. The method of claim 5, wherein said liquid extract comprises a protein concentration of about 313 ng/ml.

9. The method of claim 5, wherein said effective amount of a filtrated bacteria-free and/or yeast free liquid extract of initial fermentative kefir is administered orally.

10. The method of claim 5, wherein said malignant cells are selected from the group consisting of estrogen responsive cancer, cancer induced by oncovirus, hepatic cancer, colon cancer, prostate cancer, skin cancer and lung cancer.

11. The method of claim 10, wherein said estrogen responsive cancer is breast or uterine cancer.

12. A prophylactic composition having neutraceutical properties, which comprises a filtrated bacteria-free and/or yeast-free liquid extract of initial fermentative kefir in association with a pharmaceutically acceptable carrier.

13. The prophylactic composition of claim 12, wherein said filtrated extract is ultrafiltrated or microfiltrated.

14. The prophylactic composition of claim 13, wherein said liquid extract comprises a protein concentration of 300 ng/ml to 5000 ng/ml.

15. The prophylactic composition of claim 14, wherein said liquid extract comprises a protein concentration of about 313 ng/ml.
